(19) United States
(12) Patent Application Publication
(10) Pub. No.: US 2007/0155768 A1
(43) Pub. Date: Jul. 5, 2007

(54) Pharmaceutical composition of vinflunine which is intended for parenteral administration preparation method thereof and use of same

(76) Inventors: Elie Leverd, Castres (FR); Joel Bougaret, Francarville (FR); Marie-Dominique Ibarra, Souilhanels (FR)

Correspondence Address: BIRCH STEWART KOLASCH & BIRCH, PO BOX 747, FALLS CHURCH, VA, 22040-0747, US

(21) Appl. No.: 10/584,445

(22) PCT Filed: Dec. 17, 2004
(86) PCT No.: PCT/FR04/03287

§ 371 Date: Jun. 22, 2006

(30) Foreign Application Priority Data
Dec. 23, 2003 (FR) 0315312

(51) Int. Cl. A61K 31/4745

(52) U.S. Cl. 514/283

(57) ABSTRACT

The invention relates to a pharmaceutical composition of vinflunine in the form of a stable sterile aqueous solution of a water-soluble salt of vinflunine with a pH of between 3 and 4. The invention also relates to the method of preparing said composition and to the use thereof as a parenterally-administered medicament for the treatment of cancer.

The present invention relates to a pharmaceutical composition for the parenteral administration of vinflunine.

Study of the antineoplastic properties of the alkaloids from Vinca rosea (Apocynacea family) has already made it possible to demonstrate the advantageous activities of compounds of indole structure, for instance vincristine, vinblastine or derivatives thereof, for instance vinflunine: 20′,20′-difluoro-3′,4′-dihydrovinorelbine of formula (a) below: 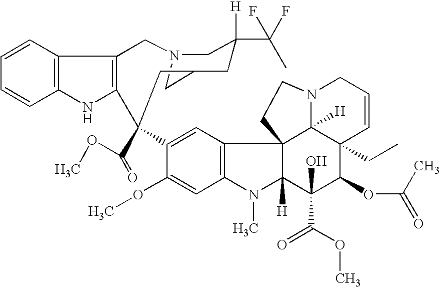
described in patent EP 0 710 240.

However, the development of injectable formulations of these active principles has always come up against problems associated with their stability in aqueous solution.

For many years, only the lyophilized form was marketed. Since it required an extemporaneous reconstitution with the contents of a solvent phial before administration, the lyophilisate presented major drawbacks associated with the hazards arising from handling it:
risk of reconstitution being performed incorrectly, during which fine droplets of product are generated, which may contaminate the person(s) performing the treatment, or the premises,
use of a poor amount of solvent or of an inappropriate amount of active principle if the pharmaceutical specialty is presented in different bottles corresponding to different unit doses.

This latter point is particularly important. It illustrates the potential possibilities of a non-therapeutic dose being administered to the patient or of exposure of the patient to an accidental overdose.

U.S. Pat. No. 4,619,935 suggested the possibility of formulating ready-to-use injectable solutions for Vinca alkaloids.

However the formulations used are complex. They comprise, in addition to the active principle:
a sugar or a sugar-based polyol, for instance mannitol,
an acetate buffer, to maintain the pH of the solution in the range 3.0-5.0 and more particularly in the range 4.4-4.8. Its molarity is between 0.02 and 0.0005 M and preferably between 0.01 and 0.002 M,
antimicrobial preserving agents.

It should be noted that, despite the stabilizing effect attributed to the acetate buffer, which makes it possible to prevent any degradation due to a change in pH caused by the decomposition of the alkaloids, the formulation that was the subject of the invention had a stability of only one year at 5° C.

The complexity of the patented formulations is increasing: patent FR 2 653 998 describes a pharmaceutical composition for parenteral use, containing an alkaloid of bis-indole type such as vincristine, vinblastine or 5′-nor-anhydrovinblastine. It is characterized in that it comprises, in aqueous solution, a zinc complex of an alkaloid salt of bis-indole type, a divalent metal gluconate and a preserving agent dissolved in an monohydric or polyhydric alcohol.

The stability indicated for these compositions is at least 24 months when they are stored in a refrigerator.

European patent EP 0 298 192 presents the favourable effect of ethylenediaminetetraacetic acid salts, in particular the sodium salt, on the stability of aqueous solutions of dimeric Vinca alkaloids. These aqueous solutions are buffered with an acetate buffer in order to maintain the pH between 3.0 and 5.5 and preferably between 4.0 and 5.0.

Under these conditions, with regard to the specifications adopted (alkaloid content of between 90% and 110% of the theoretical content), the solution remains stable for 30 months at a temperature of 2 to 8° C.

Canadian patent 2 001 643, relating to an injectable solution of vincristine, also emphasises the need to use an acetic acid/sodium acetate buffer to maintain the pH of the solution between 3.5 and 5.5, and more particularly between 4.0 and 4.5. The formulation described in the invention is stable for 18 months at 5° C., and may even be stable for 24 months at 5° C.

Vinflunine ditartrate, or 20′,20′-difluoro-3′,4′-dihyrovinorelbine L(+)-tartrate, is a white powder that must be stored at a negative temperature, below −15° C., under an atmosphere of an inert gas such as nitrogen or argon.

It has been found, entirely unexpectedly, that vinflunine ditartrate is much more stable once it is dissolved in water than in pulverulent form.

Specifically, the injectable aqueous solution is stored at a positive temperature, of between +2° C. and +8° C. This is entirely surprising since it is well known that chemical degradation reactions take place more easily in liquid medium than in the solid state.

The present invention thus relates to a vinflunine pharmaceutical composition, characterized in that it is in the form of a stable and sterile aqueous solution of a water-soluble vinflunine salt at a pH of between 3 and 4.

The subject of the invention is based on the extraordinary simplicity of the formulation, which contrasts with the compositions described in the patents initially recalled.

Advantageously, the vinflunine salt is vinflunine ditartrate.

Advantageously, the pharmaceutical composition according to the present invention is in the form of a stable, sterile and apyrogenic, ready-to-use, injectable aqueous solution.

Advantageously, the composition according to the present invention does not contain any preservatives.

In a first embodiment of the present invention, the pharmaceutical composition according to the present invention is in the form of a simple aqueous solution of vinflunine ditartrate, without addition of buffer solution. The composition thus consists of vinflunine ditartrate and water for an injectable preparation. Advantageously, the pH of this solution is equal to 3.5

In a second embodiment of the present invention, the pharmaceutical composition according to the present invention comprises a pH buffer system in order to maintain the pH between 3 and 4. Even more advantageously, the pharmaceutical composition according to the present invention consists of vinflunine ditartrate, water for an injectable preparation and a pH buffer in order to maintain the pH between 3 and 4. Advantageously, the molarity of the pH buffer system is between 0.002 M and 0.2 M.

Advantageously, the buffer system consists of an acetic acid/sodium acetate buffer or a citric acid/sodium citrate buffer.

Advantageously, the pH is obtained with acetic acid/sodium acetate or citric acid/sodium citrate buffer solutions with molarity of between 0.05 M and 0.2 M.

Even more advantageously, the pH buffer consists of the acetic acid/sodium acetate buffer and the pH of the composition is then 3.5, or the pH buffer consists of the citric acid/sodium citrate buffer and the pH of the composition is then 4.

Advantageously, the composition according to the present invention contains vinflunine ditartrate with a base vinflunine concentration of between 1 and 50 mg/ml, advantageously between 25 and 30 mg/ml and in particular 25 mg/ml or 30 mg/ml. This concentration is thus expressed as base vinflunine. The administered amount depends on the body surface area of the patients.

In one advantageous embodiment, the composition according to the present invention corresponds to one of the following formulations: 68.35 mg of vinflunine ditartrate qs 2 ml in water, or 136.70 mg of vinflunine ditartrate qs 4 ml of water, or 341.75 mg of vinflunine ditartrate qs 10 ml of water, the amount of vinflunine ditartrate corresponding, respectively, in each of the formulations to 50 mg of base vinflunine, 100 mg of base vinflunine and 250 mg of base vinflunine. These data are collated in Table 1 below.

TABLE 1
Examples of unit compositions of the aqueous solution

| Name of the components | Vinflunine unit doses | | | | | |
|---|---|---|---|---|---|---|
| Vinflunine ditartrate | 68.35 | mg | 136.70 | mg | 341.75 | mg |
| corresponding to base | 50.00 | mg | 100.00 | mg | 250.00 | mg |
| vinflunine | | | | | | |
| Water for injectable | qs 2 | ml | qs 4 | ml | qs 10 | ml |
| preparations | | | | | | |

Table 1 above shows the possibility of preparing in bottles 3 unit doses of vinflunine resulting from the distribution into different volumes of the same aqueous vinflunine ditartrate solution at a concentration of 25 mg/ml expressed in terms of base vinflunine.

In another embodiment of the invention, the composition according to the present invention remains stable for at least 36 months at 5° C.±3° C.

In one particular embodiment of the invention, the pharmaceutical composition according to the present invention is administered by intravenous perfusion, after being dissolved in perfusion solutions such as 0.9% sodium chloride or 5% glucose solutions.

The present invention thus also relates to the pharmaceutical composition according to the present invention for its use as a medicinal product, in particular for treating cancer, advantageously for a parenteral administration, advantageously via intravenous perfusion, and more advantageously during chemotherapy as an antineoplastic and antitumoral agent.

The present invention also relates to the use of a composition according to the present invention for the manufacture of a medicinal product for parenteral administration, advantageously via intravenous perfusion, which is advantageously intended for treating cancer.

The parenteral administration, especially intravenously, of a pharmaceutical vinflunine composition according to the present invention makes it possible to treat cancers that are sensitive to the action of vinflunine.

The present invention also relates to a process for preparing a composition according to the present invention, comprising the following successive steps:
(a) dissolution of the vinflunine salt in water for injectable preparations,
(b) optional addition of a pH buffer,
(c) sterilization by filtration of the bulk solution.

In one particular embodiment of the invention, the process according to the present invention comprises the additional step (d) of aseptic distribution, under a nitrogen atmosphere, of the sterile composition obtained in step (c) in a container. Advantageously, this container is chosen from glass phials, preferably of amber or colourless type I, glass bottles, preferably of amber or colourless type I equipped with an elastomer stopper and a crimped aluminium cap or any compatible ready-to-use system, for instance a prefilled syringe.

The present invention thus also relates to a packaging container containing the composition according to the present invention.

This packaging container may be chosen from glass phials preferably of amber or colourless type I, glass bottles preferably of amber or colourless type I equipped with an elastomer stopper and a crimped aluminium cap or any compatible ready-to-use system, for instance a prefilled syringe.

The examples that follow are given as non-limiting indications.

### EXAMPLE 1

### Comparison of the Stability of Vinflunine Ditartrate in Pulverulent Form with that of Vinflunine Ditartrate in Aqueous Solution (Composition According to the Present Invention)

Table 2 below shows the stability results obtained for a batch of pulverulent lyophilized vinflunine ditartrate (batch 503) and a batch of aqueous solution containing 25 mg/ml of base vinflunine (batch SB0222) manufactured with this same batch of vinflunine ditartrate, after 3 months and 6 months of storage at 25° C. The stability is monitored by observing the changes in the total amount of vinflunine-related impurities present.

TABLE 2
vinflunine ditartrate/aqueous solution stability results

| | Vinflunine ditartrate | Aqueous solution containing |
|---|---|---|
| | (batch 503) | 25 mg/ml (batch SB0222) |
| | (% impurity relative to | (% impurity relative |
| | 100% of active principle) | to 100% active principle) |
| t₀ | 1.17 | 1.23 |
| t_{3 months} | 2.75 | 1.45 |
| t_{6 months} | 3.48 | 2.00 |

After storage for 6 months at 25° C., the total amount of vinflunine-related impurities increased by:
62% in the aqueous vinflunine ditartrate solution,
197% for the pulverulent vinflunine ditartrate.

### EXAMPLE 2

### Study of Stability as a Function of the pH of the Compositions According to the Present Invention

Stability studies were Performed on Aqueous vinflunine ditartrate solutions, in a pH range of between 2.5 and 5.0 and more particularly between 3.0 and 4.0. The pH was obtained with 0.2 molar acetic acid/sodium acetate or citric acid/sodium citrate buffer solutions.

The percentage formulations used are presented in Table 3 below. They correspond to a base vinflunine concentration of 30 mg/ml.

TABLE 3
Formulations of buffered aqueous solutions

| | Compositions | | | | | |
|---|---|---|---|---|---|---|
| | BS1332 | | BS1330 | | BS1327 | |
| | (pH = 3.5) | | (pH = 3.5) | | (pH = 4.0) | |
| Vinflunine ditartrate | 4.101 | g | 4.101 | g | 4.101 | g |
| Corresponding to base | 3 | g | 3 | g | 3 | g |
| vinflunine | | | | | | |
| Glacial acetic acid | 1.185 | g | | | | |
| Sodium acetate | 0.100 | g | | | | |
| Citric acid monohydrate | | | 2.885 | g | 2.460 | g |
| Sodium citrate dihydrate | | | 1.903 | g | 2.497 | g |
| Water for injectable | qs 100 | ml | qs 100 | ml | qs 100 | ml |
| preparations | | | | | | |

The results were compared with those concerning a simple vinflunine ditartrate aqueous solution, without addition of buffer solution, stored under the same conditions. The pH of this solution is equal to 3.5.

The composition and references of the test solutions are collated in Table 4 below.

TABLE 4
Composition and reference of the test solutions

| | Formulation |
|---|---|
| Composition | reference |
| Solution at pH = 2.5 (citrate buffer) | BS 1325 |
| Solution at pH = 3 (citrate buffer) | BS 1326 |
| Solution at pH = 3.5 (citrate buffer) | BS 1330 |
| Solution at pH = 4 (citrate buffer) | BS 1327 |
| Solution at pH = 5 (citrate buffer) | BS 1328 |
| Solution at pH = 3.5 (citrate buffer) | BS 1332 |
| Unbuffered aqueous solution | BS 1331 |

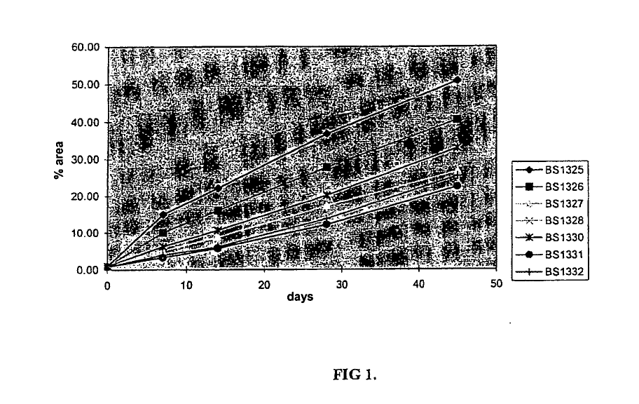

FIG. 1 shows the changes, determined by HPLC, of the content of total vinflunine-related impurities as a function of time, under severe conditions (45 days at 60° C.), for each formulation indicated in Table 3.

They are complemented by the results indicated in Table 4 below, showing the change in colour of the solutions over 7 days at 60° C.

The monitoring of the absorbance of these solutions, in the ultraviolet range, at 410 nm, reveals the appearance of vinflunine oxidation derivatives not chromatographed by HPLC.

TABLE 5
Change in absorbance

Absorbance at 410 nm

| Batch | t₀ | t_{7 days} |
|---|---|---|
| BS 1325 | 0.021 | 0.645 |
| pH = 2.5 | | |
| Citrate buffer: 0.2 M | | |
| BS 1326 | 0.020 | 0.520 |
| pH = 3.0 | | |
| Citrate buffer: 0.2 M | | |
| BS 1330 | 0.020 | 0.354 |
| pH = 3.5 | | |
| Citrate buffer: 0.2 M | | |
| BS 1327 | 0.023 | 0.346 |
| pH = 4.0 | | |
| Citrate buffer: 0.2 M | | |
| BS 1328 | 0.020 | 0.896 |
| pH = 5.0 | | |
| Citrate buffer: 0.2 M | | |
| BS 1332 | 0.021 | 0.226 |
| pH = 3.5 | | |
| Acetate buffer: 0.2 M | | |
| BS 1331 | 0.019 | 0.171 |
| pH = 3.5 | | |
| No buffer | | |

Only the unbuffered solution, pH=3.5, has an absorbance of less than 0.200 after 7 days at 60° C.

The results indicate that the stability of vinflunine is better with a pH value of between 3.0 and 4.0 but is dependent on the nature of the ions of which the buffer is composed. At pH 3.5, the acetic acid/sodium acetate buffer affords better stability than the citric acid/sodium citrate buffer. For the latter buffer, the results are better at pH 4.

It is found, entirely surprisingly, that the stability of the aqueous vinflunine ditartrate solution, at its spontaneous pH of 3.5, is better than the stability of vinflunine ditartrate aqueous solutions buffered to pH 3.5.

These good results are confirmed by the long-term results collated in Table 6 below, which indicate that the injectable aqueous vinflunine pharmaceutical composition according to the present invention may be stored for at least 36 months at 5° C.±3° C. without undergoing any substantial degradation.

TABLE 6
Stability results of the aqueous pharmaceutical
composition according to the present invention

| | | t₃ | t₆ | t₁₂ | t₂₄ | t₃₆ |
|---|---|---|---|---|---|---|
| | t₀ | ₘₒₙₜₕₛ | ₘₒₙₜₕₛ | ₘₒₙₜₕₛ | ₘₒₙₜₕₛ | ₘₒₙₜₕₛ |
| Batch CLP004 | 30.8 | 30.4 | 30.4 | 30.4 | 30.3 | 30.2 |
| Vinflunine | | | | | | |
| content in mg/ml | | | | | | |
| (theory = 30.0) | | | | | | |

1. Vinflunine pharmaceutical composition, wherein it is in the form of a stable and sterile aqueous solution of a water-soluble vinflunine salt at a pH of between 3 and 4.

2. Composition according to claim 1, wherein the vinflunine salt is vinflunine ditartrate.

3. Composition according to claim 2, wherein the composition consists of vinflunine ditartrate and water for an injectable preparation.

4. Composition according to claim 1, wherein it comprises a pH buffer system in order to maintain the pH between 3 and 4.

5. Composition according to claim 4, wherein the molarity of the pH buffer system is between 0.002 M and 0.2 M.

6. Composition according to claim 4, wherein the pH buffer system consists of an acetic acid/sodium acetate buffer or a citric acid/sodium citrate buffer.

7. Composition according to claim 2, wherein the composition contains vinflunine ditartrate with a base vinflunine concentration of between 1 and 50 mg/ml.

8. Composition according to claim 2, wherein it corresponds to one of the following formulations: 68.35 mg of vinflunine ditartrate qs 2 ml in water or 136.70 mg of vinflunine ditartrate qs 4 ml of water or 341.75 mg of vinflunine ditartrate qs 10 ml of water, the vinflunine ditartrate corresponding, respectively, to 50 mg of base vinflunine, 100 mg of base vinflunine and 250 mg of base vinflunine.

9. Composition according to claim 1, wherein it remains stable for at least 36 months at 5° C.±3° C.

10. Method for treating cancer comprising the parenteral administration of an effective amount of a composition according to claim 1 to a patient in need thereof,

11. (canceled)

12. Process for preparing a composition according to claim 1, comprising the following successive steps:
(a) dissolution of the vinflunine salt in water for injectable preparations,
(b) optional addition of a pH buffer,
(c) sterilization by filtration of the bulk solution,
(d) aseptic distribution, under a nitrogen atmosphere, of the sterile composition obtained in step (c) in the container, advantageously chosen from glass phials, glass bottles and prefilled syringes.

13. Packaging container containing the composition according to claim 1.

14. Composition according to claim 7, wherein it contains vinflunine ditartrate with a base vinflunine concentration of between 25 and 30 mg/ml.

15. Composition according to claim 14, wherein it contains vinflunine ditartrate with a base vinflunine concentration of 25 mg/ml.

16. Method for treating cancer according to claim 10, wherein the parenteral administration is via intravenous perfusion.
